(19) United States
(12) Patent Application Publication
(10) Pub. No.: US 2003/0180260 A1
(43) Pub. Date: Sep. 25, 2003

(54) Immunotherapy or treating bacterial or viral infection at mucosal surfaces with probiotics, and compositions therefor

(76) Inventors: Robert Llewellyn Clancy, New South Wales (AU); Gerald Pang, New South Wales (AU); Thomas Julius Borody, New South Wales (AU); Margaret Lorraine Dunkley, New South Wales (AU); Patricia Lynne Conway, New South Wales (AU)

Correspondence Address: FISH & RICHARDSON PC, 225 FRANKLIN ST, BOSTON, MA, 02110, US

(21) Appl. No.: 10/311,723

(22) PCT Filed: Jun. 19, 2001
(86) PCT No.: PCT/AU01/00726

(30) Foreign Application Priority Data
Jun. 19, 2000 (AU) PQ8213
Sep. 6, 2000 (AU) PQ9948

(51) Int. Cl. A61K 45/00

(52) U.S. Cl. 424/93.4

(57) ABSTRACT

Compositions and methods for therapeutic or prophylactic treatment of disorders associated with mucosal surfaces and in particular to treatment of infectious disorders at mucosal sites by enhancing non-specific mucosal immunity, especially with probiotics such as lactobacillus or mycobacterium vaccae.

### TECHNICAL FIELD

The present invention relates to compositions and methods suitable for therapeutic or prophylactic treatment of diseases associated with mucosal surfaces and in particular to treatment of infectious disorders at mucosal site by way of enhancing non-specific mucosal immunity.

### BACKGROUND ART

The last 30 years has witnessed an explosive increase in understanding of the mechanisms of mucosal protection, beginning with the recognition that mucosal immunity was partitioned from systemic immunity (with IgA as a marker), that it was driven from the gut-associated lymphoid tissue (specifically Peyer's patches), that it involved both T and B lymphocytes and that a specific recirculation of gut-derived lymphocytes between the mucosal surfaces ensured participation of all mucosal surfaces in responses generated by delivery of antigen to the Peyer's patch. Early studies focused on IgA, but gradually the key role played by T lymphocytes and the cytokines they secrete, have dominated thinking. The concept of “cytokine profiles” became important as it was shown that T cells could be characterised by the particular pattern of cytokines secreted, leading to the concept of Th1 and Th2 CD4+ve T cells. Th1 cells secreted IFN-γ while Th2 cells were characterised by IL-4 secretion. This “pattern” determined outcome and now many infection outcomes are known to be influenced by the pattern of cytokines secreted.

The above focuses on specific immunity initiated by particular antigens. The non-specific immune response “sits” on, and operates through, an array of cells and molecules which are powerful effector mechanisms operating without the specificity gained through antigen receptors.

The value in health promotion of a range of gut microbes (probiotics) taken in a variety of food or formulation forms has been recognised for some time. Claims to the value of these probiotics are mainly non-specific and without scientific support, and are largely based on clinical impression. The probiotics are thought to promote health via reconstitution of what is presumed to be beneficial normal flora. Use of probiotics for treatment of certain specific intestinal conditions has been reported. However, the frequently exaggerated claims serve to reduce credibility rather than consolidate therapeutic benefits.

Changing patterns of proneness to mucosal disease (eg. allergy, infection) are increasingly being linked to microbe/gut mucosal “experience”, in areas which include changing incidence of allergy and asthma linked to environment “sterility” and altered gut bacterial flora, and reduced mucosal infection and allergy in infants given avirulent E. coli.

There is therefore a need for improved preparations and methods for prophylaxis or therapy of mucosal disorders such as infections, allergy and the like. There is also a need for preparations and methods which can be used for prevention of reactivation of, or reinfection with, viruses and for the treatment of conditions and syndromes associated with viral infection or reinfection.

It is an object of the present invention to overcome or ameliorate at least some of the disadvantages of the prior art, or to provide a useful alternative.

### SUMMARY OF THE INVENTION

The present invention is in part based on the observation that certain microorganisms, in particular lactobacilli, can prime the mucosal surfaces by inducing a particular cytokine “pattern”, thus creating conditions unfavourable to microbial colonisation, and/or microbial pathogenesis, and in part on the novel demonstration of non-antigen activated cells migrating within the common mucosal system.

The microorganisms useful in the practice of the present invention may or may not have traditional probiotic effects, but they will be able to alter the cytokine pattern or balance, or induce a Th1-type cellular response. For convenience the useful microorganisms or their components may also be referred to herein as probiotics, whether or not they in fact have a probiotic effect. It is intended that this term (“probiotic”) includes in its scope other adjuvant agents capable of inducing a Th1-type cellular response.

According to a first aspect there is provided a method of prophylactic or therapeutic treatment of chronic or acute infection, or of undesirable microbial colonisation, of a mucosal surface, other than intestinal mucosal surface, comprising the administration of an effective amount of a probiotic, or a probiotic-containing composition, to a subject in need thereof.

In this embodiment of the invention the mucosal surface is preferably selected from the group consisting of oral, nasopharyngeal, respiratory, gastric, reproductive and glandular. The infection or colonisation can be acute or chronic and may be bacterial, fungal or viral. It will be understood that chronic viral infection will include certain syndromes which may have origins in viral infection, such as for example chronic fatigue syndrome and the like.

According to a second aspect there is provided a method of prophylactic or therapeutic treatment of a chronic or acute disorder of mucosal surface of the respiratory tract, comprising the administration of an effective amount of a probiotic, or a probiotic-containing composition, to a subject in need thereof.

Preferably the respiratory tract mucosal surface is the upper respiratory tract mucosal surface and even more preferably it is oral or lung mucosa.

Preferably the probiotic, or a probiotic-containing composition, is administered to the gastric or to the intestinal mucosal surface.

According to a third aspect there is provided a method of prophylactic or therapeutic treatment of a chronic or acute disorder of a mucosal surface caused by disturbance in cytokine balance or lack of an appropriate T cell immune response, comprising the administration of an effective amount of a probiotic, or a probiotic-containing composition, to a subject in need thereof.

Preferably the mucosal surface is selected from the group consisting of oral, nasopharyngeal, respiratory, gastric, intestinal, reproductive and glandular.

A useful marker for assessment of cytokine balance is one or more of interferon-γ (IFN-γ), interleukin-4 (IL-4) and interleukin-12 (IL-12). However, other cytokines known as markers for either Th1 or Th2 cellular responses are also useful for this purpose.

It is preferred that the probiotic is a bacterium, for example one which can be selected from, but not limited to, lactic acid bacteria, Mycobacterium species or Bifidobacterium species. Even more preferred is the use of Lactobacillus acidophilus (L. acidophilus), Lactobacillus fermentum (L. fermentum) or Mycobacterium vaccae (M. vaccae), or parts thereof which are capable of inducing the Th1 cellular response. Specially preferred is L. acidophilus. L. acidophilus, L. fermentum or M. vaccae may be used live or as an inactivated preparation, as long as they are capable of inducing the Th1 response. For preference L. acidophilus and L. fermentum is used as a live preparation. It is considered that other bacteria would also be suitable as probiotics as herein defined (whether they have probiotic effect or not), for example the well known adjuvating bacteria such as for example L. casei, L. plantarum, L. rhamnosus, Bifidobecterium breve and the like.

The required dosage amount will vary according to the nature of the mucosal surface disorder, whether used prophylactically or therapeutically and the type of organism or neoplasm involved. The treatment parameters as well as the required dosage can be easily assessed by those skilled in the art. The preferred dosage of the probiotic, when the probiotic is a whole live probiotic bacterium, is from about 1×10⁸ to about 1×10¹² organisms.

The probiotic may be administered in conjunction with one or more antibiotics or one or more other pharmaceutically active agents. The probiotic may be administered prior to, simultaneously with or subsequent to antibiotic therapy or therapy with other active agents.

Preferably, the mucosal surface disorder is a bacterial infection such as for example infection by Pseudomonas species, Streptococcus species, Staphylococcus species, Candida species, Helicobacter species or Haemophilus species. Even more preferred are non-typable Haemophilus influenzae, Pseudomonas aeruginosa, Streptococcus pneumoniae, Staphylococcus albus, Staphylococcus aureus, Candida albicans and/or Helicobacter pylori. The mucosal disorder may also be inappropriate colonisation by bacteria or other microorganisms. For example the mucosal disorder may involve chronic or acute colonisation or infection by viruses such as Epstein-Barr virus (EBV), cytomegalovirus (CMV), Herpes viruses and the like. It would be clear to those skilled in the are that the treatment of infections and/or colonisation by other viruses can also be achieved with the methods and compositions of the present invention.

Preferably, the subject in need of treatment is selected from the group consisting of individuals having high risk of infection. However, it will be recognised that the present treatments are suitably employed in prophylaxis of mucosal disorders in any subject. For example, the treatment methods of the present invention may be suitably administered to subjects exposed to a variety of stressors which have an impact on the subjects immune status and thus predisposes them to infection. For example fatigue and/or physical stressors such as commonly encountered by athletes, predisposes these subjects to respiratory tract infections. This seems to be caused by impairment in the immune status of mucosal surfaces, in particular the secretion of IgA.

Administration of the probiotic preparations, such as those described herein, prior to, during and/or after exercise or training may restore their immune status, particularly that of the mucosal surfaces of the respiratory tract, thus combating or preventing infection.

Preferably, the probiotic or probiotic-containing composition is in tablet or capsule form. However, it will be clear to those skilled in the art that the probiotic composition may be in a liquid or other forms of solid preparations and may also be present in a food source such as a yoghurt or other dairy product, or similar non-dairy products based for example on soy.

The treatment may involve administration of a probiotic, or of a probiotic-containing composition, to a site which is distal to the mucosal surface having the disorder. For example the probiotic, or the probiotic-containing composition, can be administered to the intestinal mucosal surface in the treatment of nasopharyngeal, gastric or upper respiratory tract mucosal infection.

According to a fourth aspect there is provided a method of altering cytokine balance at a mucosal surface in a subject comprising the administration of an effective amount of a probiotic, or of a probiotic-containing composition, to a subject in need thereof.

According to a fifth aspect there is provided a method of inducing a Th1 cellular immune response at a mucosal surface in a subject comprising the administration of an effective amount of a probiotic, or of a probiotic-containing composition, to a subject in need thereof.

According to a sixth aspect there is provided a method of enhancing the secretion of interferon-γ at a mucosal surface in a subject comprising the administration of an effective amount of a probiotic, or of a probiotic-containing composition, to a subject in need thereof.

According to a seventh aspect there is provided a method of reducing the secretion of interleukin-4 at a mucosal surface in a subject comprising the administration of an effective amount of a probiotic, or of a probiotic-containing composition, to a subject in need thereof.

According to an eighth aspect there is provided a method of restoring normal cytokine balance at a mucosal surface in a subject comprising the administration of an effective amount of a probiotic, or of a probiotic-containing composition, to a subject in need thereof.

According to a ninth aspect there is provided a method of priming mucosal surface for immunotherapy comprising the administration of an effective amount of a probiotic, or of a probiotic-containing composition, to a subject in need thereof.

Preferably the immunotherapy consists in administration of a therapeutic or prophylactic vaccine. Also preferred is a procedure whereby the probiotic, or the probiotic-containing composition, is administered before or simultaneously with the vaccine. The administration of the probiotic, or of the probiotic-containing composition, may be continued for a period after the vaccine has been administered.

According to a tenth aspect there is provided a pharmaceutical composition suitable for prophylactic or therapeutic treatment of chronic or acute disorder of a mucosal surface comprising an effective amount of a probiotic, or of a probiotic-containing composition.

It is preferred that the probiotic is a bacterium, for example one which can be selected from, but not limited to, lactic acid bacteria, Mycobacterium species or Bifidobacterium species. Even more preferred is the use of Lactobacillus acidophilus (L. acidophilus), Lactobacillus fermentum (L. fermentum) or Mycobacterium vaccae (M. vaccae), or parts thereof which are capable of inducing the Th1 cellular response. Specially preferred is L. acidophilus. L. acidophilus, L. fermentum or M. vaccae may be used live or as an inactivated preparation, as long as they are capable of inducing the Th1 response. For preference L. acidophilus and L. fermentum is used as a live preparation. It is considered that other bacteria would also be suitable as probiotics as herein defined (whether they have probiotic effect or not), for example the well known adjuvating bacteria such as for example L. casei, L. Plantarum, L. rhamnosus, Bifidobecterium breve and the like.

Preferably the pharmaceutical composition includes viable organisms, however it will be understood that killed organisms may also be used. Further, single organism preparations or preparations containing multiple organisms are envisaged. The organism(s) can be either intact or disrupted.

The preferred content of the probiotic, when the probiotic is a whole live probiotic bacterium, is one that will deliver a dosage from about 1×10⁸ to about 1×10¹² organisms.

The preferred formulation of the composition is in a solid dosage form, such as for example tablet or capsule. It will be understood however that it may be formulated in the form of a food product, for example soy-based or dairy-based product.

According to an eleventh aspect there is provided a method of prophylactic or therapeutic treatment of a symptom and/or syndrome associated with chronic or acute infection, or with undesirable microbial colonisation or reactivation, of a mucosal surface, comprising the administration of an effective amount of an agent capable of non-specific activation of the common mucosal system to a subject in need thereof.

Preferably the symptom and/or syndrome to be treated is chronic fatigue syndrome.

The preferred agent is a probiotic, or a probiotic-containing composition. Also preferred is that the probiotic is, or the probiotic-containing composition comprises, viable intact organisms.

Preferably the subject to be treated is selected from the group consisting of an EBV positive athlete, a subject with sudden onset CFS, a subject with protracted fatigue following exercise, a subject with low level of salivary IgA or IgA1 and a subject with documented infectious mononucleosis.

### BRIEF DESCRIPTION OF FIGURES

FIG. 1: IL-4 and IFN-γ production following feeding with Lactobacillus acidophilus

FIG. 2: Secretion of NO into saliva of animals fed Lactobacillus acidophilus following challenge with Candida albicans

FIG. 3: Effect of probiotic on IL-12 production following challenge with Candida albicans

FIG. 4: Effect of probiotic on ILK production following challenge with Candida albicans

FIG. 5: Resistant to infection in animals fed L. acidophilus.

FIG. 6: Effect of probiotic on salivary IFN-γ following challenge with Candida albicans

FIG. 7: Effect of probiotic on cervical lymph node IFN-γ following challenge with Candida albicans

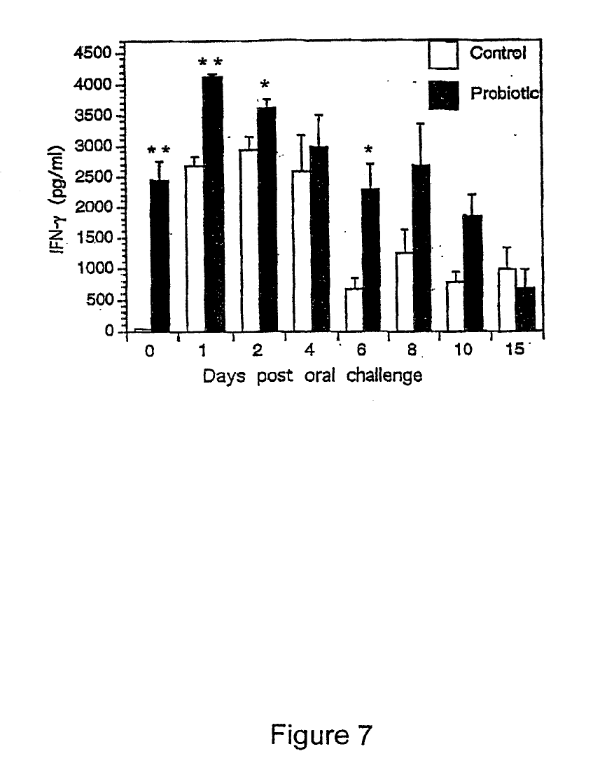

FIG. 8: Salivary IFN-γ following challenge with C. albicans.

FIG. 9: Effect of treatment with L-MLNA on clearance of C. albicans in mice

### DESCRIPTIOON OF THE PREFERRED EMBODIMENTS

The current invention is about the manipulation of cells and molecules that participate in both these systems, to “prime” mucosal surfaces to produce Th1 cytokines in response to “danger”. The concept that is developed is that certain bacteria, specially lactobacilli, populate the gut of subjects exposed to high density pathogens. Our studies have shown that these bacteria influence the cytokine pattern towards a Th1 response. The activated and IFN-γ producing T cells populate the mucosal surfaces and their regional lymph nodes, setting up a containing armament awaiting “danger”.

The methods and compositions of the present invention can also be used effectively in the treatment of acute and chronic viral infections. In particular the treatment of chronic Epstein-Barr virus (EBV), cytomegalovirus (CMV) and other herpes-type virus infection, which are ubiquitous in the population and are associated with numerous symptoms and diseases, are envisaged as particularly useful applications. Also envisaged as a useful application of the compositions and methods of the present invention is the treatment of disorders which may have origins in or are triggered by viral infections which are prevalent in the population and which can be debilitating but have no known or no well defined treatment protocol, such as for example chronic fatigue syndrome (“CFS”).

Substantial number of patients currently diagnosed as “CFS” reflect repeatedly activated EBV (or similar herpes group virus infection), due to impaired mucosal immunity. This can be monitored through measurement of salivary IgA1 or INF-γ, or PCR detected free virus. Characteristic (but not limiting) clinical features of those likely to respond to probiotic therapy includes onset of “viral illness”, leaving a state of chronic relapsing fatigue. Acute deterioration occurs with “infection”, usually noted as a sore throat, or excessive exercise.

Viral infection need not cause immediately recognisable symptoms and may be dormant in otherwise fit and healthy subjects for a significant periods of time, but may reactivate once a trigger is provided. For example, in athletes undergoing intensive training or exercise impaired performance may be caused by reduced mucosal containment of EBV, which leads to reactivation and excretion of virus, which in turn leads to impaired performance and/or fatigue. Thus, athletes who are EBV positive and have impaired performances, specially if low salivary IgA or IgA1 is detected (for example less than 50 mg/100 ml), could benefit from treatment with preparations of the present invention.

Of course the preparations and methods of the present invention can also be applied to other subjects, for example patients diagnosed as “CFS”, where a virus (usually EBV but can be CMV, Ross River virus and the like) is reactivated and the fatigue is particularly initiated by exercise. However, any patient with documented infectious mononucleosis not getting better within several weeks and left with fatigue, any patient with protracted fatigue following clinical (serological) evidence of viral illness or subjects with sudden onset CFS, with recurrent sore throats, with significant exacerbation of fatigue following exercise, or with low level of salivary IgA or IgA1 (eg. less than 50 mg/100 ml), could benefit from treatment with the preparations of the present invention.

It is envisaged that the administration of the probiotic or compositions containing the probiotic, will also assist in the treatment of symptoms and/or disorders described above in relation to viral infections.

The probiotics have the effect on mucosal surfaces distal to the site of administration of the probiotic. Thus, in broad terms the present invention is concerned with a probiotic product, in particular a product which is or includes lactobacilli, but may include other bacteria or combinations of bacteria, or indeed other adjuvants capable of inducing a Th1-type cellular response, which can be administered as a bolus or preferably regularly fed to maintain optimal mucosal protection of all mucosal surfaces through priming of mucosal T cells or maintaining the primed state of the cells. Such T cells contain INF-γ and can be triggered to release INF-γ by non specific mechanisms. Therefore, other agents capable of activating non-specifically the common mucosal system may also be advantageously used in the preparations and methods of the present invention.

Further, the probiotics of the present invention may be used in conjunction with other treatments, to enhance or assist in their efficacy. For example, approximately 20% of patients treated with antibiotics for H. pylori infection fail to eradicate the organism. This resistance to antibiotic therapy may be due to a shift towards a Th0 response (ie, less IFN-γ and more IL-4). Administration of a probiotic prior to, in conjunction with or subsequent to antibiotic therapy can be beneficial by switching back to a more dominant Th1 response and thus supplementing or assisting the antibiotic therapy to eradicate the organism in such patients.

Administration of probiotic can therefore be used for prevention or therapy of mucosal infections, colonisation of mucosal surfaces with abnormal or inappropriate organisms and reinfection with or reactivation of viruses.

The invention will now be described more particularly with reference to non-limiting examples.

### EXAMPLES

### Example 1

### Effect of Probiotic Bacteria on Th1/Th2 Cytokine Response

To determine whether probiotic bacteria down-regulate Th2 and up-regulate Th1 cytokine response, C57/BI6 mice were fed intragastrically using a feeding needle, various numbers of Lactobacillus acidophilus (obtained from University of New South Wales, School of Microbiology and Immunology Culture Collection, Sydney, Australia) on consecutive days for 2 weeks after which they were sensitised with 8 μg of ovalbumin (OVA) and aluminium hydroxide in 0.2 mL phosphate-buffered saline administered by peritoneal injection. The mice were further fed ten times with L. acidophilus every two days for two weeks before they were sacrificed. Lymphocytes were isolated by teasing spleens through a sieve, washed with PBS, and resuspended at 10×10⁶ One mL aliquots of the cell suspension were dispensed into wells of a 24-well flat-bottomed microtitre plate and stimulated with OVA (5 μg/mL). After incubation for 4 days the supernatants were collected and assayed for IL-4 and IFN-γ production by standard ELISA techniques using IL-4 or IFN-γ monoclonal antibody pairs.

Briefly, wells of a 24-well microtitre plate were coated with a capture anti-IL-4 antibody. After incubation at room temperature for 1 hr, the wells were washed and biotinylated anti-IL-4 antibody was added to each well. Following incubation for a further 1 hr, the wells were washed and strepavidin-peroxidase conjugate was added to each well. After incubation for 30 mins, the wells were washed and then TMB substrate was added. The colour development was read at 450/620 nm in an ELISA plate reader. The level of IL-4 in unknown samples was quantitated by interpolation using a standard curve. A similar procedure was used for measurement of IFN-γ.

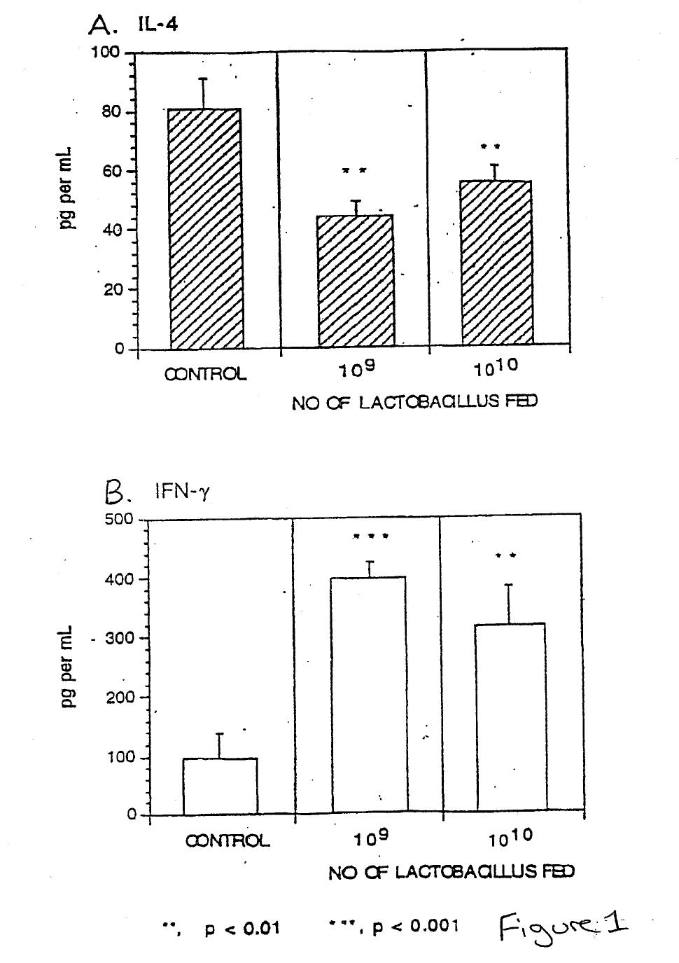

The results shown in FIG. 1A and B demonstrate that feeding L. acidophilus resulted in the suppression of IL-4 production in dose-dependent manner (FIG. 1A) whereas the production of IFN-γ was enhanced (FIG. 1B).

### Example 2

### Effect of Probiotic Bacteria on Nitric Oxide (NO) Secretion in Saliva

DBA/2 mice (n=3 to 5 per group) were fed Lactobacillus acidophilus and then challenged with Candida albicans as described in Example 3 below. At various times following oral infusion, saliva was collected after injection with pilocarpine to stimulate saliva flow. The concentration of NO in saliva was determined by the Griess reaction according to the method by Gree et al (1982) Anal. Biochem 126:131-138.

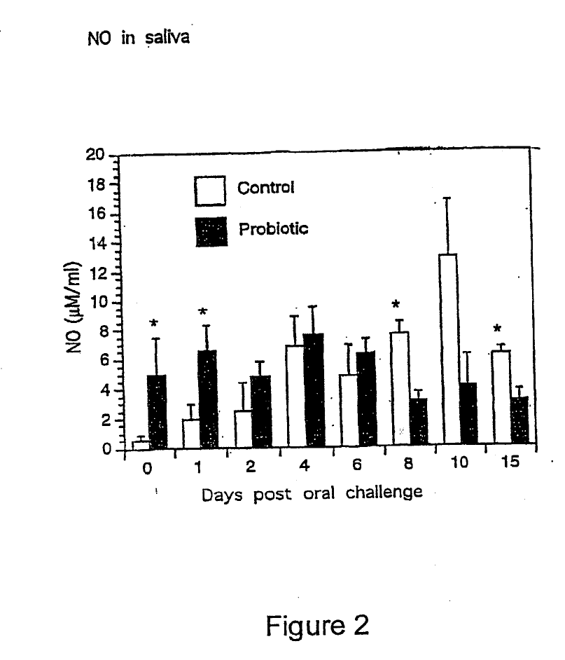

The data demonstrate that secretion of NO into saliva of animals fed Lactobacillus acidophilus (oral administration of 5×10⁹ CFU) was increased early after challenge with Candida albicans but was significantly reduced at a later stage (FIG. 2).

### Example 3

### Resistance to C. albicans Following Feeding with Probiotic Bacteria

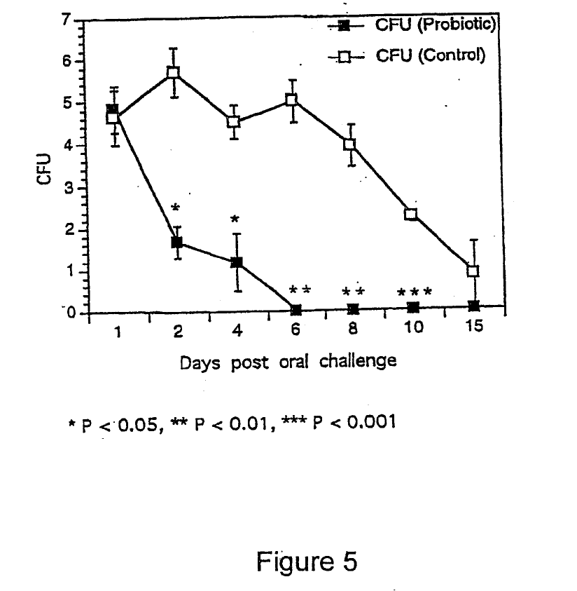

DBA/2 mice H2d male mice (n=3-5 per group) were fed intragastrically every 2 days for 2 weeks with 5×10⁹ L. acidophilus in 0.2 mL PBS. One day after the last feed mice were challenged with 10⁸ Candida albicans blastospores by swabbing the oral cavity using a fine-tip sterile swab. At various days after infection the number of C. albicans in the oral mucosa was determined. The oral cavity (cheek, tongue and soft palate) was completely swabbed using a fine-tipped cotton swab. The cotton swab was placed in 1 mL PBS and the yeast cells were resuspended by mixing in a vortex mixer before culture of serial 10-fold dilutions on Sabourand dextrose agar supplemented with chloramphenicol at 37° C. The results were expressed as CFU/mL (FIG. 5)

Mice fed L. acidophilus were more resistant to infection at all time points compared to control mice fed PBS. By day 6 the yeasts were almost completely cleared from mice fed L. acidophilus.

### Example 4

### IL-4, IFN-γ and IL-12 Production

A further study on responses to Candida albicans infection was conducted in the model described in Example 3.

Candida antigen was prepared from freshly cultured Candida albicans by sonication in a MSE Soniprep. The sonicate was centrifuged for 10 min at 2000 g after which time the supernatant was collected and dialysed against PBS. After protein estimation, the solution was filtered sterilised and stored at −20° C. until needed.

A single cell suspension of cervical lymph node (CLN) cells was prepared by teasing lymph node tissue through a sieve. The cells were collected in RPMI 1640 medium supplemented with 10% foetal calf serum (FCS), washed twice by centrifugation and cultured at 4×10⁶ cells per well in the presence of 2.5 μg/mL of Candida antigen in a 24-well plate for 3 days.

The culture supernatants were collected and assayed for IL-4, IL-12 and IFN-γ by ELISA using matched antibody pairs and recombinant cytokines as standards (Pharmingen, San Diego, Calif.). The sensitivity of the cytokine ELISA was 31 μg/mL and the results were expressed in net amounts from which the background was subtracted.

Samples of saliva were obtained according to the procedure described in example 2.

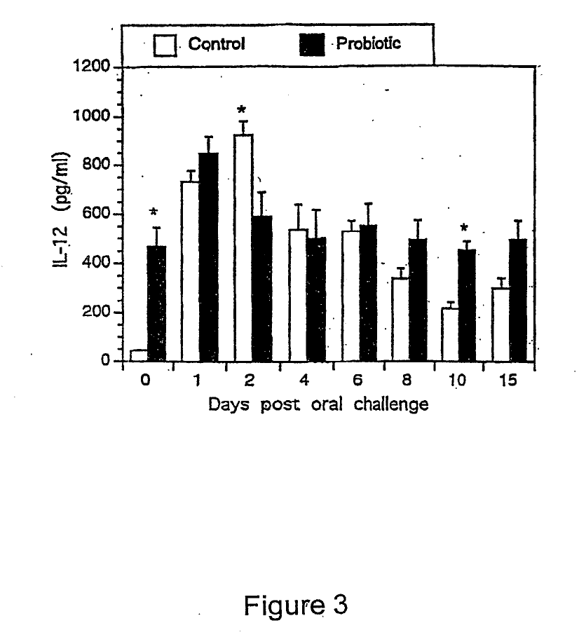

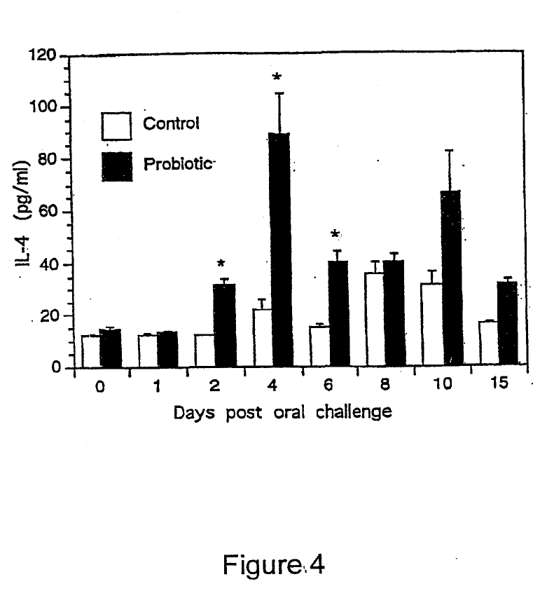

The results of IL-4 and IL-12 study are shown in FIG. 3 (IL-12) and FIG. 4 (IL-4).

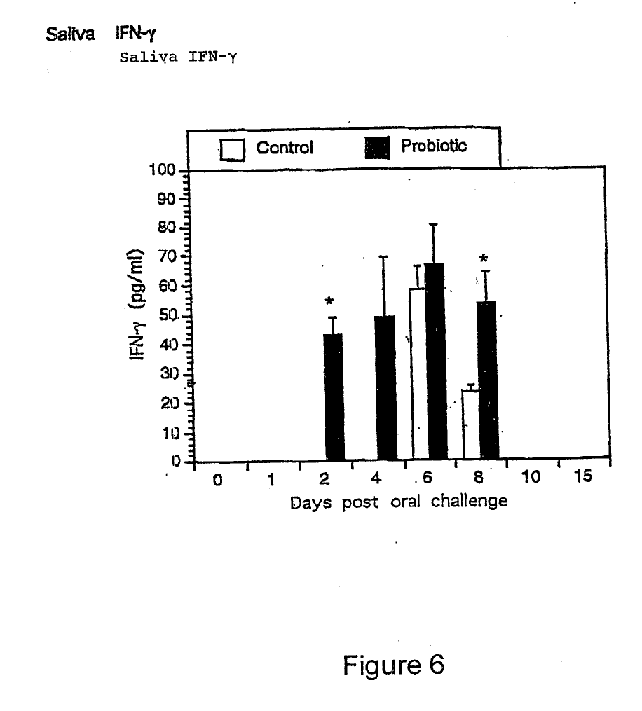

The results of the IFN-γ study are shown in FIGS. 6 (salivary IFN-γ) and 7 (cervical lymph node IFN-γ).

The control data shows a small poorly sustained response to oral infection with Candida albicans, with no detectable IFN-γ until day 6 of the infection. The data in mice fed L. acidophilus indicates that there is no IFN-γ response in uninfected mice but that there is an early response (day 2) when mice were challenged with C. albicans. Further, there was a more sustained IFN-γ response after challenge with C. albicans than in the controls not fed L. acidophilus. This primary effect can still be seen in the nodes at day 15 and in saliva at day 8.

Not wishing to be bound by any particular mechanism of action, it seems that the mucosal surfaces are primed by the administration of Lactobacillus to secrete IFN-γ following a challenge and in this model IFN-γ operates to prevent conversion of the pathogen, Candida albicans, to the mycelial (invasive) form of the fungus and enhances cellular immunity and nitric oxide production.

### Example 5

### Role of IFN-γ and NO in Resistance to Infection

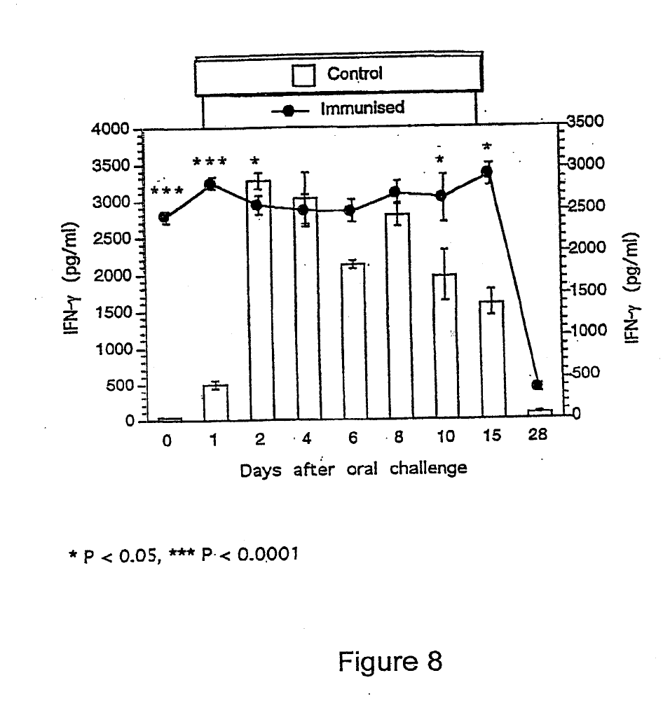

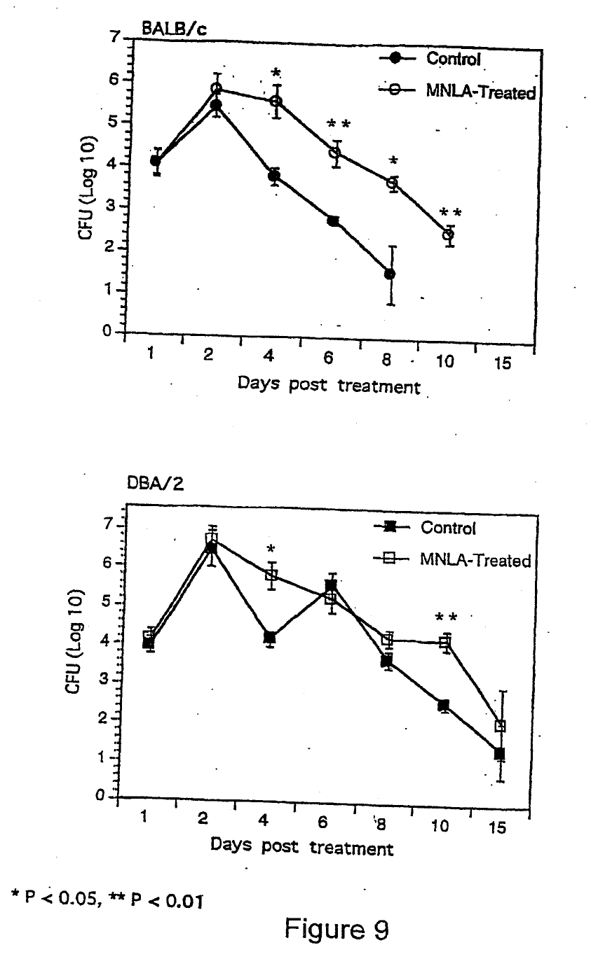

To identify the immune parameters of protection, the levels of IFN-γ were determined at various times following infection with C. albicans. As shown in FIG. 8, there was a marked increase in the levels of IFN-γ in saliva immediately and on day 1 after challenge with C albicans compared with unimmunised mice. The levels were sustained over 15 days with significantly higher levels detected on day 10 and day 15 compared with those in unimmunised mice. Since nitric oxide production is associated with host defence in parasitic infection, quantitation of NO was performed following infection in two mouse strains sharing the same H2d MHC haplotype. In this experiment mice were infected with C albicans and then followed by ip injection with an inhibitor of NO synthase, by injecting NG-monomethyl-L-arginine monoacetate (MNLA) daily for 3 days after which time the clearance rate of yeasts was determined. As shown in FIG. 9, mice treated with MNLA had delayed clearance of yeasts at various time points in the two mouse strains compared with untreated mice, indicating that reduction in NO production is associated with resistance.

### Example 6

### Enhanced Clearance of Non-Typable H influenzae from the Lungs of Rats Fed L acidophilus

DA rats (200-250 gm, 8-10 weeks old, Animal Resource Centre, Perth, Wash.) were fed intragastrically L acidophilus in 0.75 mL PBS (2.5×10¹⁰ per rat) or PBS alone every 2 days for 7 days at which time the rats were immuniised with formalin-killed H influenzae (5×10 per rat) administered intralumenally (see groups below). Rats continued to be fed every 2 days for 2 weeks and then boosted with 50 μL of formalin killed H influenzae (5×10⁸ per rat) administered by the intratracheal route. After further feeding with L acidophilus for a further 7 days, the rats were challenged with 50 μL of live 5×10⁸ H influenzae in the lung.

### Immunisation Groups (5 Rats/Group):

1 PBS (intralumenal)

2H influenzae (intralumenal)+H influenzae (intra-tracheal boost)

3 PBS (intralumenal)+L. acidophilus fed every 2 days

4H influenzae (intralumenal)+H influenzae (intratracheal boost)+L. acidophilus fed every 2 days

After 4 hrs post challenge, the rats were sacrificed and the levels of colonisation in the lung was examined in bronchial lavage (BAL) and lung homogenate (LH). The level of clearance was determined by a plating serial 10-fold dilutions of the lavage fluid or lung homogenate onto chocolate agar plates. The results were expressed as number of colony forming units (CFU.

The results are shown in Table 1. Rats fed L acidophilus and immunised with killed H influenzae were more resistance to infection H influenzae in the lungs. Furthermore, rats fed orally with repeated doses of L acidophilus were more resistant to infection than rats given a single bolus of L acidophilus, suggesting that enhanced clearance may be due to increased colonisation in the gut with L acidophilus following repeated feeding.

TABLE 1
Increased clearance of H influenzae in the lungs of rats fed
L acidophilus

| Groups | BAL (CFU/mL × 10⁸) | LH (CFU/mL × 10⁶) |
|---|---|---|
| PBS | 26 ± 7.5 | 90.2 ± 29 |
| H influenzae + H | 4.6 ± 3.0 | 4.9 ± 1.2 |
| influenzae boost | | |
| PBS + L. acidophilus | 0.7 ± 0.3 | 12.5 ± 5.7 |
| H influenzae + H | 0.19 ± 0.1 | 3.2 ± 2.1 |
| influenzae boost + L. | | |
| acidophilus | | |

### Example 7

### Effect of Lactobacillus acidophilus (VRI 011) on Translocation of Salmonella typhimurium in Mice Following Immunisation with Heat Killed Salmonella Vaccine.

Translocation of gram-negative bacteria across the gut epithelium can occur especially in subjects following post-operative surgery or gastrointestinal infection. Left untreated it can lead to endotoxiemia. In this example, the effect of feeding L acidophilus on the translocation of gut pathogen Salmonella typhimurium is examined.

Female BALB/c mice 6-8 weeks old were obtained from the Animal Resource Centre, Perth, Wash. Lactobacillus acidophilus strain (VRI 011) and the pathogen Salmonella tyhimurium were obtained from the School of Microbiology and Immunology Type Culture Collection, University of New South Wales.

Mice were given 5 doses of L acidophilus (VRI 011) (1×10⁸ cfu per dose) or PBS over a 2 week period after which time mice were immunised intragastrically with a killed oral Salmonella vaccine. Two weeks after immunisation, the mice were sacrificed and the spleens collected for enumeration of L acidophilus and S tyhimurium.

Mice fed L acidophilus (VRI 011) before or simulatneously with a killed oral S typhimurium vaccine have better protection against translocation of S tyhpimuirum following live challenge via the oral route compared with mice fed PBS (Table 1). Immunisation with a killed oral vaccine led to decreased translocation of S typhimurium to the spleen but it was further enhanced by feeding L acidophilus. Thus translocation of pathogenic bacteria through the gut epithelium can be prevented by oral administration of probiotic.

### Example 8

### Effect of Lactobacillus fermentum on Translocation of Pathogenic S tyhphimurium from the Gut of Mice Vaccinated with Killed Oral Salmonella Vaccine

BALB/c female mice (6-8 weeks old) were fed 10⁸ L fermentum VRI012 (obtained from University of New South Wales, School of Microbiology and Immunology Culture Collection, Sydney, Australia) before or co-administered with 10⁸ killed S typhimurium vaccine four times over a 2 week period. Two weeks after oral administration, the mice were challenged with live S typhimurium administered by the oral route. After sacrifice, the level of colonisation with S typhimurium in the spleen was determined. Table 2 showed that low levels of translocation occurred in mice fed L fermentum prior to immunisation or co-administered with S typhimurium compared with mice fed PBS.

TABLE 2
L fermentum VRI012 enhanced the inhibition of translocation of
S typhimurium in mice immunsed with killed S typhimurium vaccine.

| Group treatment | Log CFU/g wet wt of spleed (±SEM) |
|---|---|
| PBS (Control) | 10.12 ± 0.54 |
| Killed S typhimurium | 5.5 ± 0.27 p < 0.05 vs Control |
| L fermentum KLD ( before) | 3.8 ± 0.12 p < 0.05 vs Control |
| L fermentum KLD (co- | 4.5 ± 0.22 p < 0.05 vs Control |
| administered) | |

### Example 9

### Effect of L. fermentum on Clearance of NTHI Bacteria

Groups of 6 male, specific pathogen free, dark agouti (DA) rats were given a single dose of PBS or live L. fermentum (2.5×10¹⁰) directly into the duodenal lumen after exposure of the intestine by laparotomy. Twenty-one days later the rats were infected intratracheally with 5×10⁸ live Non-typeable Haemophilus influenzae (NTHi). Four hours later the rats were killed by pentobarbitone overdose. The lungs were lavaged with 10 mL PBS to obtain broncho-alveolar lavage fluid (BAL). The lavaged lungs were then homogenised in 10 mL of PBS. Twenty microlitres of ten-fold serial dilutions of BAL and lung homogenate (LH) were plated on chocolate agar plates and incubated in a 10% CO2 incubator at 37° C. overnight. Colonies were counted and the number of live bacteria in the BAL and LH preparations determined. The recovery of live NTHi bacteria, presented as colony forming units (CFU), was as follows:

TABLE 3

| Rats dosed with: | BAL CFU (10⁶) | LH (10⁶) |
|---|---|---|
| PBS | 3.67 ± 1.30 | 103.5 ± 34.2 |
| L. fermentum | 0.49 ± 0.19 | 0.82 ± 0.37 p = 0.017* |

*When compared to PBS group

These data show that a single dose of L. fermentum delivered to the gut causes enhanced clearance of a subsequent acute NTHi respiratory infection.

### Example 10

### Effect of L acidophilus on Clearance of H. pylori

Two groups of 5 female C57BL/6 mice were infected with H. pylon (Sydney strain 1) by administration of approx. 10⁹ live H. pylori on three consecutive days (days 1, 2 and 3) by gavage. On days 28, 30, 32, 35, 37 and 39 mice were dosed by gavage with live L. acidophilus (5×10⁹ in 0.2 mL water) or water (control group).

Twenty-one days later (day 50) mice were killed by pentobarbitone overdose (administered intra-peritoneally) and their stomachs removed. The stomach was dissected into two equal halves and one half was placed in 1 mL of deionised water and homogenised. Serial 10-fold dilutions of stomach homogenate were plated out on chocolate agar plates containing fungizone, vancomycin, bacitracin and nalidixic acid. After incubation for 3 days at 37° C. under microaerophilic conditions, the colonies were counted and the total number of live H. pylori in the original stomach homogenate determined. The results for recovery of live H. pylori in the stomach, expressed as colony-forming units (CFU), are shown in the following table.

TABLE 4

| | Number of H. pylori in stomach homogenate |
|---|---|
| Mice dosed with: | (10⁴ CFU) |
| Deionised water | 252 ± 44 |
| L. acidophilus in water | 105 ± 14 |

The two groups are significantly different (P = 0.013)

These results show that mice dosed with L. acidophilus have a lower level of H. pylori infection of the stomach than do control mice. This is an example of a therapeutic effect of L. acidophilus on a pre-established H. pylori infection.

### Example 11

### Anti-Bacterial Therapy (Metronidazole) of Helicobacter pylori Infection in Mice Pre-Treated with Probiotic

H. pylori infected mice (5 per group) were treated with Lactobacillus acidophilus (1×10⁹ per animal) by oral feeding 3 times a week for 2 weeks prior to treatment with Metronidazole (0.08 mg/animal) for 1 week.

The following results demonstrate that highly significant eradication of bacteria from the stomach was noted in mice pretreated with probiotic and followed by antibiotic therapy (70%, p=0.009), those treated with probiotic alone (58%, p=0.013), and those treatment with metronidazole alone (55%, p, 0.025), when compared with the saline-treated control group.

TABLE 6

| | H. pylori colonisation | |
|---|---|---|
| Treatment Group | (×10⁵ CFU/animal) | % eradication |
| Saline | 25 ± 4.3 | 0 |
| L acidophilus | 10.5 ± 1.4 | 58^{a} |
| Metronidazole | 11.4 ± 2.5 | 55^{b} |
| L acidophilus + | 7.5 ± 2.6 | 70^{c} |
| Metronidazole | | |

^{a}p < 0.01;
^{b}p < 0.02;
^{c}p < 0.005 compared with control values

The above examples demonstrate that probiotic bacteria, by driving a particular cytokine balance (from gut derived but mucosally re-located T cells), create an environment less favourable for particular microbial growth.

Although the present invention has been described with reference to preferred embodiments it will be understood that variations in keeping with the inventive concept described herein are also contemplated.

The claims defining the invention are as follows:

1. A method of prophylactic or therapeutic treatment of chronic or acute infection, or of undesirable microbial colonisation, of an affected mucosal surface in an immunocompetant subject other than in the gastrointestinal tract, comprising contacting another mucosal surface of the subject remote from the affected mucosal surface with an effective amount of a probiotic for producing a non-specific cellular immune response for treatment of the affected mucosal surface and which is characterised by expression of a cytokine profile associated with a Th1 response or shift in cytokine expression toward a Th1 cytokine profile, and wherein the probiotic is other than Mycobacterium vaccae.

2. A method according to claim 1, wherein the mucosal surface for being treated is selected from the group consisting of the nasopharangeal, respiratory, reproductive and glandular mucosa.

3. A method according to claim 2, wherein the mucosal surface for being treated is selected from the group consisting of the nasopharangeal and respiratory mucosa.

4. A method according to claim 1, wherein the mucosal surface for being treated is the mucosal surface of the lung.

5. A method according to any one of claims 1 to 4, wherein the immune response is characterised by the expression of a cytokine profile associated with a Th1 response.

6. A method according to any one of claims 1 to 4, wherein the immune response is characterised by upregulated expression of one or more cytokines associated with a Th1 cytokine profile.

7. A method according to any one of claims 1 to 6, wherein the non-specific cellular immune response is generated in the intestine.

8. A method according to any one of claims 1 to 7, wherein the chronic or acute infection is a viral infection.

9. A method according to any one of claims 1 to 8, wherein the probiotic comprises bacteria.

10. A method according to any one of claim 9, wherein the bacteria are viable bacteria.

11. A method according to any one of claims 1 to 10, wherein the probiotic is selected from the group consisting of Lactobacillus acidophilus, Lactobacillus fermentum and Lactobacillus casei.

12. A method of prophylactic or therapeutic treatment of chronic or acute infection, or of undesirable microbial colonisation, of an affected mucosal surface in a subject other than in the gastrointestinal tract, comprising contacting another mucosal surface of the subject remote form tie affected mucosal surface with an effective amount of a probiotic for producing a non-specific cellular immune response for treatment of the affected mucosal surface and which is characterised by expression of a cytokine profile associated with a Th1 response or shift in cytokine expression toward a Th1 cytokine profile, and wherein the infection or microbial colonisation is non-fungal and the probiotic is other than Mycobacterium vaccae.

13. A method according to claim 12, wherein the mucosal surface for being treated is selected from the group consisting of the nasopharangeal, respiratory, reproductive and glandular mucosa.

14. A method according to claim 13, wherein the mucosal surface for being treated is selected from the group consisting of the nasopharangeal and respiratory mucosa.

15. A method according to claim 12, where the mucosal surface for being treated is the mucosal surface of the lung.

16. A method according to any one of claims 12 to 15, wherein the immune response is characterised by the expression of a cytokine profile associated with a Th1 response.

17. A method according to any one of claims 12 to 15, wherein the immune response is characterised by upregulated expression of one or more cytokines associated with a Th1 cytokine profile.

18. A method according to any one of claims 12 to 17, wherein the nonspecific cellular immune response is generated in the intestine.

19. A method according to any one of claims 12 to 18, wherein the chronic or acute infection is a viral infection.

20. A method according to any one of claims 12 to 19, wherein the probiotic comprises bacteria.

21. A method according to claim 20, wherein the bacteria arc viable bacteria.

22. A method according to any one of claims 12 to 21, wherein the probiotic is selected from the group consisting of Lactobacillus acidophilus, Lactobacillus fermentum and Lactobacillus casei.

23. A method according to claim 20 or 21, wherein the effective amount of the probiotic is in a range of from about 1×10⁸ bacteria to 1×10¹² bacteria.

24. A method of prophylactic or therapeutic treatment of a viral infection of an affected mucosal surface in a subject, comprising contacting another mucosal surface of the subject remote from the affected mucosa surface with an effective amount of a probiotic for producing a non-specific cellular immune response for treatment of the affected mucosal surface and which is characterised by expression of a cytokine profile associated with a Th1 response or shift in cytokine expression toward a Th1 cytokine profile, and wherein the probiotic is other than Mycobacterium vaccae.

25. A method according to claim 24, wherein the mucosal surface for being treated is other than the gastrointestinal mucosa.

26. A method according to claim 24, wherein the mucosal surface for being treat is selected from tae group consisting of the oral nasopharangeal, respiratory, reproductive and glandular mucosa.

27. A method according to claim 26, wherein the mucosal surface for being treated is selected from the group consisting of the nasopharangeal and respiratory mucosa.

28. A method according to claim 24, wherein the mucosal surface for being treated is the mucosal surface of the respiratory tract.

29. A method according to claim 24, wherein the mucosal surface to be treated is mucosal surface of the lung.

30. A method according to any one of claims 24 to 29, wherein the viral infection is selected from an infection by Epstein-Barr virus (EBV), cytomegalovirus (CMV), Ross River virus (RRV), herpes-type virus and the like.

31. A method of prophylactic or therapeutic treatment of a chronic or acute disorder of an affected mucosal surface in a subject, comprising contacting another mucosal surface of the subject remote from the affected mucosal surface with an effective amount of a probiotic for producing a non-specific cellular immune response for treatment of the affected mucosal surface and which is characterised by expression of a cytokine profile associated with a Th1 response or shift in cytokine expression toward a Th1 cytokine profile, wherein the probiotic is other than Mycobacterium vaccae and the chronic or acute disorder is other than an infection of the affected mucosal surface by a Candida species.

32. A method according to claim 31, wherein the mucosal surface for being treated is selected from the group consisting of the nasopharangeal, respiratory, reproductive and glandular mucosa.

33. A method according to claim 32, wherein the mucosal surface for being treated is selected from the group consisting of the nasopharangeal and respiratory mucosa.

34. A method according to claim 31, wherein the mucosal surface for being treated is the mucosal surface of the lung.

35. A method according to any one of claims 31 to 34, wherein the immune response is characterised by the expression of a cytokine profile associated with a Th1 response.

36. A method according to any one of claims 31 to 34, wherein the immune response is characterised by upregulated expression of one or more cytokines associated with a Th1 cytokine profile.

37. A method according to any one of claims 31 to 36, wherein non-specific cellular immune response is generated in the intestine.

38. A method according to any one of claims 31 to 37, wherein the chronic or acute disorder is a viral infection.

39. A method according to any one of claims 31 to 38, wherein the probiotic comprises bacteria.

40. A method according to claim 39, wherein the bacteria are viable bacteria.

41. A method according to any one of claims 31 to 40, wherein the disorder is an infection or inappropriate microbial colonisation.

42. A method according to claim 41, wherein the infection is a bacterial infection caused by one or more bacteria selected from the group consisting of Pseudomonas species, Streptococcus species, Staphylococcus species and Haemophilus species.

43. A method according to claim 42, wherein the one or more bacteria are selected from the group consisting of non-table Haemophilus influenzae, Pseudomonas aeruginosa, Streptococcus pneumnoniae, Staphylococcus albus, and Staphylococcus aureus.

44. A method according to claim 41, wherein the disorder is an infection or inappropriate colonisation by a virus.

45. A method according to claim 44, wherein the virus is selected from the group consisting of Epstein-Barr virus (EBV), cytomegalovirus (CMV), Ross River virus (RRV), herpes-type virus and the like.

46. A method according to claim 31, wherein the disorder is chronic fatigue syndrome (CFS).

47. A method according to any one of claims 31 to 46 further comprising administering to the subject one or more pharmaceutically active agents prior to, simultaneously with, or subsequent to, the probiotic.

48. A method of enhancing the secretion of interferon-γ at a mucosal surface in a prophylactic or therapeutic treatment of a subject in need thereof, comprising contacting a mucosal surface of tie subject remote from the mucosal surface at which the secretion of interferon-γ is to be enhanced, with an effective amount of a probiotic for inducing expression of a cytokine profile associated with a Th1 response or upregulating the secretion of the interferon-γ, and wherein the probiotic is other than Mycobacterium vaccae.

49. A method of reducing the secretion of interleukin-4 at a mucosal surface in a prophylactic or therapeutic treatment of a subject in need thereof, sin contacting a mucosal surface of the subject remote from the mucosal ace at which the secretion of interleukin-4 is to be reduced, with an effective amount of a probiotic for inducing a shift in cytokine expression toward a Th1 response, and wherein the probiotic is other than Mycobacterium vaccae.

50. A method of priming a mucosal surface of a subject, other than in the intestinal tract, in immunotherapy wherein the method comprises contacting a mucosal surface of the subject remote from the mucosal surface for being primed, with an effective amount of a probiotic for producing a non-specific cellular immune response characterised by expression of a cytokine profile associated with a Th1 response or shift in cytokine expression toward a Th1 response, and wherein the probiotic is other than Mycobacterium vaccae.

51. A method according to claim 50, wherein the shift in cytokine expression toward a Th1 response comprises upregulated secretion of one or more cytokines associated with a Th1 response.

52. A method of prophylactic or therapeutic treatment of a syndrome associated with chronic or acute infection, or with microbial colonisation or reactivation, of an affected mucosal surface in a subject other than in the intestinal tract, comprising contacting a mucosal surface remote from the affected mucosal surface with an effective amount of a probiotic for producing a non-specific cellular immune response for treatment of the affected mucosal surface and which is characterised by expression of a cytokine profile associated with a Th1 response or shift in balance toward a Th1 cytokine profile, wherein the probiotic is other than Mycobacterium vaccae.

53. A method according to claim 52, wherein the syndrome is chronic fatigue syndrome.

54. A method according to claim 52 or 53, wherein the immune response is characterised by upregulated expression of one or more cytokines associated with a Th1 cytokine profile.

55. A method according to any one of claims 52 to 54, wherein the probiotic comprises bacteria.

56. A method according to claim 55, wherein the bacteria are viable bacteria.

57. A method according to any one of claims 52 to 56, wherein the probiotic is selected from the group consisting of Lactobacillus acidophilus, Lactobacillus fermentum and Lactobacillus casei.

58. A method of prophylactic or therapeutic treatment of a condition or disorder associated with chronic or acute infection or with microbial colonisation or reactivation, of an affected mucosal surface in a subject comprising contacting a mucosal surface remote from the affected mucosa surface to be treated with an effective amount of a probiotic for producing a non-specific cellular immune response for treatment of the affected mucosal surface and which is characterised by expression of a cytokine profile associated with a Th1 response or shift in a cytokine expression toward a Th1 cytokine profile, and wherein the subject is selected from the group consisting of an EBV positive athlete, a subject with sudden onset chronic fatigue syndrome, a subject with protracted fatigue following exercise, a subject with low level salivary IgA or IgA1 and a subject with documented mononucleosis, and wherein the probiotic is other than Mycobacterium vaccae.

59. A method according to claim 58, wherein the immune response is characterised by upregulated expression of one or more cytokines associated with a Th1 cytokine profile.

60. A method according to claim 58 or 59, wherein the probiotic comprises bacteria.

61. A method according to claim 60, wherein the bacteria are viable bacteria.

62. A method according to any one of claims 58 to 61, wherein the probiotic is selected from the group consisting of Lactobacillus acidophilus, Lactobacillus fermentum and Lactobacillus casei.

63. A pharmaceutical composition when used in a method as defined in any one of claims 1 to 62, and incorporating the probiotic.

64. A pharmaceutical composition according to claim 63, wherein the composition contains about 10⁸ to about 10¹² CFU.

65. A pharmaceutical composition according to claim 64, wherein the composition contains about 10¹¹ CFU.

66. A pharmaceutical composition according to any one of claims 63 to 65, wherein the composition is in a solid dosage form.

67. A pharmaceutical composition according to claim 66, wherein the dosage form is a tablet or a capsule.
